(19) United States
(12) Patent Application Publication
(10) Pub. No.: US 2018/0310883 A1
(43) Pub. Date: Nov. 1, 2018

(54) METHOD AND DEVICE FOR RECORDING MOVEMENT IN A CONTINUOUS AREA

(71) Applicant: WEARABLE TECHNOLOGIES PTY LTD, Balwyn North, Victoria (AU)

(72) Inventors: Mitchell Jared FINLAYSON, Balwyn North (AU); Brenton Grant FINLAYSON, Balwyn North (AU); Samuel David ROSENGARTEN, Balwyn North (AU)

(73) Assignee: WEARABLE TECHNOLOGIES PTY LTD, Balwyn North, Victoria (AU)

(21) Appl. No.: 15/768,398

(22) PCT Filed: Oct. 17, 2016
(86) PCT No.: PCT/AU2016/000358

§ 371 Date: Apr. 13, 2018

(30) Foreign Application Priority Data
Oct. 16, 2015 (AU) 2015904233

(51) Int. Cl. A61B 5/00; A61B 5/11

(52) U.S. Cl. CPC A61B 5/6833; A61B 5/1116; A61B 5/1118; A61B 5/746; A61B 5/7228; A61B 2562/0271; A61B 2505/09; A61B 2560/0223; A61B 2560/045; A61B 2562/0219; A61B 2562/0261; A61B 5/0004

(57) ABSTRACT

A device for recording movement and/or posture in a continuous area comprising an elastic therapeutic tape substrate for application to a body part, the tape substrate comprising an elastic two dimensional conductor along which an electrical current can flow to produce deformation data comprising change in resistance and/or capacitance as the elastic two dimensional conductor changes geometry in response to movement is described. The device may further comprise a data collection unit which receives the deformation data. Also described are a method and system for recording movement and/or posture in a continuous area comprising applying the elastic therapeutic tape substrate to a body part and producing deformation data comprising change in resistance and/or capacitance as the elastic two dimensional conductor changes geometry in response to movement to thereby record the movement in the continuous area.

### FIELD OF THE INVENTION

The present invention relates to a method, device and system for recording movement in a continuous area. More particularly, the invention relates to a method and device for recording movement in a continuous area in order to monitor activity and/or assess biomechanics.

### BACKGROUND TO THE INVENTION

In order to improve sporting or athletic performance, biomechanical studies may be performed to analyse performance and determine weaknesses or areas where improvement may be obtained.

U.S. Pat. No. 7,627,451 to Vock et al., and Assigned to Apple Inc., describes a movement monitoring device (MMD) that can be coupled to an adhesive strip. The MMD has a detector attached to a processor, communications port and battery. The detector is used to sense movement of the MMD. Individual MMDs may be attached at discrete points on a person such as, on each foot and hand, head, knee and chest. The data collected may be used to observe contact such as, when hit by a punch or kick, and to compare one strike to another.

US Patent Publication 2011/0306921, the publication of U.S. patent application Ser. No. 13/157,156, teaches a device for multimodal stimulation of living tissue. The device has an elastic matrix containing an electrically conductive region capable of being in communication with a power source and the tissue. Tailorable electrical signal input is used to strengthen muscles. Active electrodes may be placed to provide TENS therapy and passive electrodes may be placed to provide EMG data for kinesiology. The electrodes are positioned in selectable locations on the matrix and provide point source data. The elastic matrix provides some support that is applied to the tissue and compliantly responds to force resultant from motion. This publication reports that the device described provides multimodal stimulation of continuous kinaesthetic and proprioceptive stimuli coupled with periodic electrical nerve-training to strengthen the patient and build self-regulating body awareness.

US Patent Publication 2007/00830096, the publication of U.S. patent application Ser. No. 10/581,476, teaches a wearable system for the monitoring of vital signs that has point source electrodes connected to an electronic device that transmits data to a remote system. The remote system correlates that received data to a series of indexes to generate alert signals. A related feedback signal can be sent back to the user to enhance personal self-management and enhance personal self-management and reaction to potential health risks.

There is a need for improved methods and devices for recording movement.

### SUMMARY OF THE INVENTION

The present invention is directed to a method, device and system for recording movement in a continuous area.

In a broad form, the invention relates to a method and device for recording movement and posture in a continuous area in order to measure activity and/or assess biomechanics for prevention, reduction and/or rehabilitation of an injury, disease or other condition or improvement or modification of athletic performance.

A preferred advantage of the method and device of the present invention is that injury, disease or other conditions may be prevented and/or reduced and the data collected may be used in rehabilitation. Another preferred advantage of the method and device of the present invention, is that the data collected may be used to improve and/or modify athletic performance or posture that may be utilised to increase an athlete's likelihood of success. In situations where athletic performance is to be modified, an athlete's career may be lengthened by eliminating or reducing a movement that causes or is likely to cause injury.

In a first aspect, the present invention provides a device for recording movement and/or posture in a continuous area comprising:
an elastic therapeutic tape substrate for application to a body part, the tape substrate comprising an elastic two dimensional conductor along which an electrical current can flow to produce deformation data comprising change in resistance and/or capacitance as the elastic two dimensional conductor changes geometry in response to movement.

In one embodiment of the first aspect, the device further comprises a data collection unit which receives the deformation data.

In another embodiment of the first aspect, the elastic two-dimensional conductor comprises one or more connectors for joining to respective one or more connectors comprised on the data collection unit.

In still another embodiment of the first aspect, the data collection unit further comprises one or more additional sensor.

In yet another embodiment of the first aspect, the data collection unit further comprises an alarm.

In another embodiment of the first aspect, the alarm may be activated when an undesired movement is detected from the received deformation data. The alarm may be activated on one or more of a data collection unit; a personal computing device in communication with the data collection unit; and a server computer in communication with the data collection unit.

In still embodiment of the first aspect, the undesired movement may be detected when received deformation data matches reference deformation data associated with the undesired movement. The reference deformation data may be stored on a memory comprised in the data collection unit or a memory comprised on a personal computing device or a server computer.

In a second aspect, the present invention provides a method for recording movement and/or posture in a continuous area comprising;
applying an elastic therapeutic tape substrate to a body part, the elastic therapeutic tape substrate comprising an elastic two dimensional conductor along which an electrical current can flow; and
producing deformation data comprising change in resistance and/or capacitance as the elastic two dimensional conductor changes geometry in response to movement to thereby record the movement in the continuous area.

In one embodiment of the second aspect, the method further comprises sending the recorded deformation data to a data collection unit.

The method of the second aspect may further comprise joining one or more connectors on the elastic two-dimensional conductor to respective one or more connectors on the data collection unit.

The method of the second aspect may further comprise receiving data from one or more additional sensor.

The method of the second aspect may further comprise activating an alarm when an undesired movement is detected from the received deformation data.

According to the second aspect, the detection of the undesired movement may comprise matching received deformation data with reference deformation data associated with the undesired movement.

According to the second aspect, the reference deformation data may be stored on a memory comprised in the data collection unit or a memory comprised on a personal computing device or a server computer. When a match is detected, the alarm may be activated in the data collection unit by the data collection unit or in response to a signal from a personal computing device or a server computer.

In one embodiment of the second aspect, a plurality of elastic therapeutic tape substrates may be applied at different positions on one or more body parts.

The plurality of elastic therapeutic substrates may be connected to a same data collection unit or may comprise respective individual data collection units.

In a third aspect, the invention provides a kit comprising one or more devices according to the first aspect and one or more data collection unit.

In a fourth aspect, the present invention provides a system for recording movement and/or posture in a continuous area comprising;
an elastic therapeutic tape substrate for application to a body part; and
an elastic two dimensional conductor comprised on the elastic therapeutic tape substrate along which an electrical current can flow for producing deformation data comprising change in resistance and/or capacitance as the elastic two dimensional conductor changes geometry in response to movement.

In one embodiment of the fourth aspect, the system further comprises a data collection unit for receiving the deformation data.

The system of the fourth aspect may further comprise respective connectors on the elastic two-dimensional conductor and the data collection unit for interconnection.

The system of the fourth aspect may further comprise one or more additional sensor for generating additional data.

The system of the fourth aspect may further comprise an alarm for notifying when an undesired movement is detected from the received deformation data.

According to the fourth aspect, the detection of the undesired movement may comprise matching received deformation data with reference deformation data associated with the undesired movement.

According to the fourth aspect, the reference deformation data may be stored on a memory comprised in the data collection unit or a memory comprised on a personal computing device or a server computer. When a match is detected, the alarm may be activated in the data collection unit by the data collection unit or in response to a signal from a personal computing device or a server computer.

In one embodiment of the fourth aspect, a plurality of elastic therapeutic tape substrates may be applied at different positions on one or more body parts.

The plurality of elastic therapeutic substrates of the fourth aspect may be connected to a same data collection unit or may comprise respective individual data collection units.

In one embodiment of any above aspect, the conductor connectors comprise magnetic studs comprised on the tape substrate.

In another embodiment of any above aspect, the data collection unit connectors comprise magnetic studs comprised on the tape substrate.

In yet another embodiment of any above aspect, the conductor connectors and the data collection unit connectors may be inter-fitting.

In still another embodiment of any above aspect, the data collection unit further comprises one or more power supply; a microcontroller; and two or more connectors for connection to the elastic elongate or sheet conductor.

In another embodiment of any above aspect, the data collection unit further comprises a case.

In yet another embodiment of any above aspect, the data collection unit is small enough to be worn long term.

In still another embodiment of any above aspect, the data collection unit is connected to personal computing device to provide a visual representation of movement and position of the body on the personal computing device. The visual representation may be in the form of an avatar, gauge, graph, chart or any other visually meaning representation.

In another embodiment of any above aspect, the data collection comprises or utilises GPS (Global Positioning System) data received from the personal computing device to pattern movement behaviours. The GPS data may be used to gain greater understanding of body movement across speeds and location of the user.

In yet another embodiment, the data collection unit detects and analyses movement patterns.

In still another embodiment of any above aspect, the data collection unit may measure and learn movement patterns. The learning of movement patterns and their detection from received deformation data may be used to stop, warn and/or prevent harm to the user before they are aware of the potential damage.

In another embodiment, data collected by the data collection unit may be transmitted and/or stored on a server. The stored data may be viewed by a health or professional, performance professional or by the wearer. The health professional, performance professional or wearer may have access to an interface on a personal computing device. The health professional or performance professional can interpret the data which may be shown in a table, graph, chart or other visual method and provide feedback to the wearer of the device.

In still another embodiment of any above aspect, the one or more additional sensor comprises one or more of a temperature sensor; three axis accelerometer; three axis gyroscope and/or a strain gauge. The one or more additional sensor may be used to determine the type and extent of activities being performed.

In yet another embodiment of any above aspect, the alarm comprises a visual alarm;

aural alarm and/or vibratory alarm. The visual alarm may comprise a light emitting diode or similar light source. The aural alarm may comprise a speaker emitting an alert sound. The vibratory alarm or physical alarm vibration may emanate from the data collection unit, or from a personal computing device.

In one embodiment of any above aspect, the data collection unit may comprise a wireless link to the personal computing device.

According to any above aspect, the personal computing device may comprise a smart phone; a tablet computer; or other portable computer.

In another embodiment of any above aspects, the data collection unit is connected to the personal computing device wirelessly. The wireless connection may comprise a Bluetooth wireless connection. The Bluetooth wireless connection may comprise a low energy Bluetooth wireless connection.

In yet another embodiment of any above aspect the personal computing device may be connected to the server computer through a network. The connection of the personal computing device to the network may comprise a USB, Wi-Fi, internet or other such connection.

In still another embodiment of any above aspect, the device, method, kit or system may be for use or when used to measure activity and/or assess biomechanics for prevention, reduction and/or rehabilitation of an injury, disease or other condition or improvement or modification of athletic performance or posture.

The disease may comprise a degenerative disease. The degenerative disease may comprise one or more of Alzheimer's disease; Amyotrophic Lateral Sclerosis (ALS), a.k.a., Lou Gehrig's Disease; Osteoarthritis; Atherosclerosis; Chronic obstructive Pulmonary Disease (COPD); Chronic traumatic encephalopathy; Diabetes; Ehlers-Danlos Syndrome; Essential tremor; Friedreich's ataxia; Huntington's Disease; Marfan's Syndrome; Multiple sclerosis; Multiple system atrophy; Muscular dystrophy; Niemann Pick disease; Osteoporosis; Parkinson's Disease; Progressive supranuclear palsy; Rheumatoid Arthritis; Motor neuron Conditions; Metabolic Muscle Conditions; Conditions of the peripheral nerve; Conditions of the Neuromuscular Junction; Neuromuscular Myopathies; Chronic Pain; Chronic Swelling; Bursitis; Fibromyalgia; Back Pain; Scoliosis; Tendonitis; Degenerative Disk Disease.

In another embodiment of any above aspect, the body part may comprise a human or non-human body part. The non-human body part may comprise a performance animal body part such as, an equine or a canine body part.

In one embodiment of any above aspect, the deformation data allows determination of change in geometry of the elastic two dimensional conductor. The change in geometry may comprise one or more of elongation and retraction.

In another embodiment of any above, the elastic therapeutic tape comprises an elastic cotton strip having an acrylic adhesive. The elastic therapeutic tape may also comprise a synthetic polymer such as, nylon, and/or other natural or synthetic materials.

In still another embodiment of any above, the elastic two dimensional conductor comprises a stretchable conductive fabric; a stretchable polymeric material; or other conductive material. In one suitable embodiment the elastic two dimensional conductor comprises a silver plated, nylon containing elastic fibre fabric.

In yet embodiment conductive material comprising the conductor is woven into the tape substrate. The weave may comprise any pattern including U-shaped and zig-zag patterns.

In another embodiment of any above aspect, the elastic two dimensional conductor comprises or consists of graphene. The graphene may be sprayed and/or painted on.

In another embodiment, the elastic two dimensional conductor comprises or consists of gold, silver, nickel, conductive carbon or any other suitable material capable of changing its electrical properties to detect deformation.

In one embodiment of any above aspect, the elastic two dimensional conductor is attached to or integrated within the elastic therapeutic tape.

In another embodiment of any above aspect the elastic two dimensional conductor comprises an elongate or a sheet shape.

In still another embodiment of any above aspect, the elastic two dimensional conductor comprises a shape comprising two or more proximally located ends.

In another embodiment of any above aspect, the elastic two dimensional conductor comprises a shape comprising two opposed ends. Both conductor connectors may be disposed at one opposed end. When the elastic two dimensional conductor comprises a shape comprising two opposed ends, it may comprise a sheet or strip shape.

The shape may comprise a weave of conductive material in any shape or pattern such to collect required deformation data.

The shape may comprise one or more materials capable of changing electrical properties when deformed in a shape or pattern so as to collect required deformation data.

Examples of shapes comprising two or more proximally located ends include one or more of a U-shape; a V-shape; an arch; an ogive. The shape may comprise two or more shapes comprising proximally located ends. Each of the two or more shapes may be connected in parallel or series. Each of the two or more shapes may be located side by side; arranged in a pattern or nested.

The shape may comprise an incomplete circle or oval. The incomplete circle or oval may comprise a missing portion of its circumference.

Examples of arch shapes include a triangular arch; a round arch; a rowlock arch; a segmental arch; an unequal round arch; a lancet arch; an equilateral pointed arch; a shouldered flat arch; a trefoil arch; a horseshoe arch; a three centered arch; an elliptical arch; an inflexed arch; an ogee arch; a reverse ogee arch; a tudor arch; a parabolic arch; a stilted arch; a bell arch; a cusped arch; and a Florentine arch. Examples of ogives are a standard ogive; an equilateral ogive; a lancet ogive; a dropped ogive and a Moorish ogive.

The elastic two dimensional conductor may comprise a length and a width. The length and/or width may comprise at least two points from which data are collected. The length and width is any suitable extent to provide useful data.

The elastic two dimensional conductor may comprise a length of at least 1, 2, 3, 4 or 5 centimeters. Length may be as long as required to provide useful data from the area of interest.

The elastic two dimensional conductor may comprise a width of at least 1, 2, 3, 4 or 5 centimeters. Width may be as wide as required to provide useful data from the area of interest.

Where the terms “comprise”, comprises”, “comprising”, “include”, “includes”, “included” or “including” are used in this specification, they are to be interpreted as specifying the presence of the stated features, integers, steps or components referred to, but not to preclude the presence or addition of one or more other feature, integer, step, component or group thereof.

Further, any prior art reference or statement provided in the specification is not to be taken as an admission that such art constitutes, or is to be understood as constituting, part of the common general knowledge.

### BRIEF DESCRIPTION OF THE FIGURES

In order that the present invention may be readily understood and put into practical effect, reference will now be made to the accompanying illustrations, wherein like reference numerals refer to like features and wherein:

FIG. 1: shows one embodiment of a device according to the invention.

FIGS. 2A and 2B shows one embodiment of a personal computing device suitable for use according to one embodiment of the invention.

FIG. 3: shows one embodiment of a method according to the invention.

FIGS. 4 and 5: show one embodiment of a prototype according to the invention.

FIGS. 6A and 6B: show front (A) and rear (B) views of one embodiment of a data collection unit according to the invention

FIG. 7: shows another embodiment of a method according to the invention.

FIGS. 8A and 8B: show perspective (A) and side (B) views of a working prototype according to one embodiment of the invention.

FIGS. 9A and 9B: show perspective (A) and side (B) views of a data collection unit according to one embodiment of the invention.

FIGS. 10A and 10B: show perspective (A) and side (B) views of a data collection unit according to one embodiment of the invention.

### DETAILED DESCRIPTION OF THE INVENTION

The following description refers to specific embodiments of the present invention and is in no way intended to limit the scope of the present invention to those specific embodiments.

In one embodiment, the invention provides an electronic wearable device that incorporates the benefits of elastic therapeutic tape whilst being capable of detecting and/or analysing motion with the aim of providing useful feedback to a user and/or clinician for injury prevention, reduction and/or rehabilitation as well as for improving and/or modifying athletic performance or posture.

As used herein “a substrate” is a supporting material on which a circuit may be formed or fabricated.

As used herein “two dimensional” means have an extent in a length direction and a width direction and excludes a single point.

As used herein “elongate” means a shape, of any length, that is relatively longer than it is broad, but which is not a point.

As used herein “sheet” means any broad shape which is not a point.

As used herein “elastic” means capable of returning to its original length and shape after being stretched, deformed, compressed, or expanded.

As used herein “athletic performance” includes both results based performance such as speed and strength and also the manner or quality of the movement executed. For example, an increase in speed is an example of a results based improved athletic performance and a reduction or elimination of a non-desired movement is an example of an improved manner or quality of athletic performance. An improved manner or quality may or may not lead to an improved results based performance.

As will be clear from the context, as used herein “geometry” means shape and size.

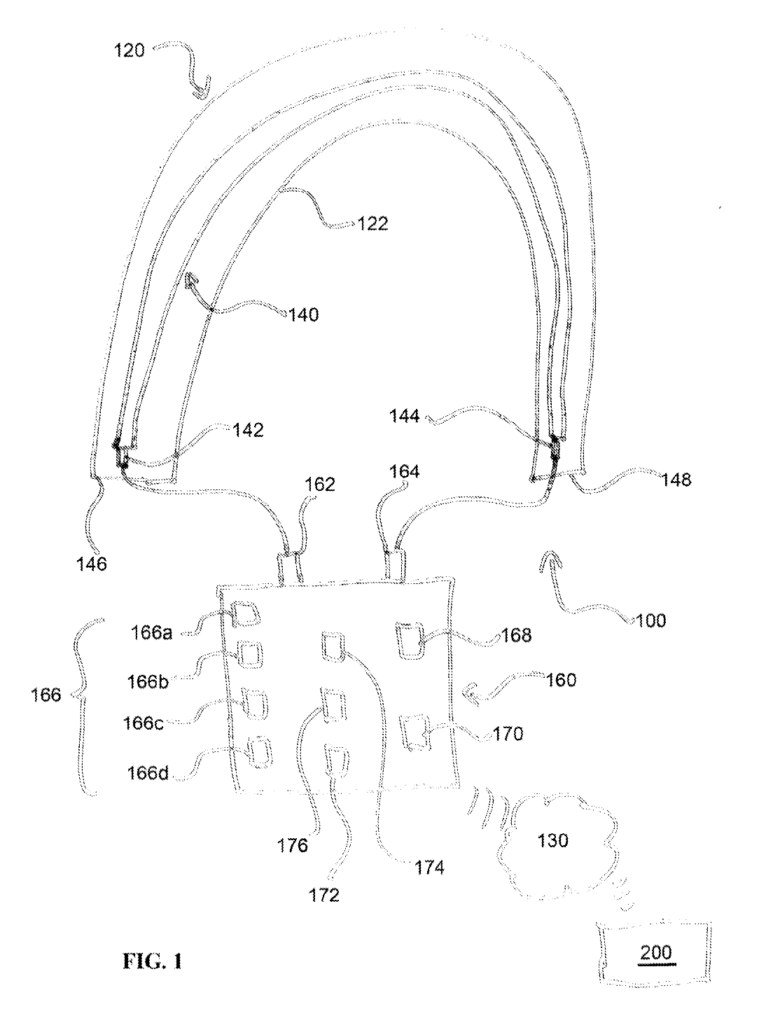

FIG. 1 shows one embodiment of a device 100 for sensing movement and/or posture according to the invention. Device 100 comprises an elastic therapeutic tape substrate 120 and an elastic two dimensional conductor 140.

So that it may be applied to a body part, tape substrate 120 comprises an adhesive 122 on the side to be affixed to the body part. Any suitable adhesive may be used such as an acrylic adhesive; a natural rubber latex adhesive; a silicone adhesive; a hydrocolloid adhesive; a hydrogel adhesive; or a polyurethane adhesive. From the teaching herein person of skill in the art is readily able to select a suitable adhesive.

In one form, elastic tape substrate 120 comprises an elastic cotton strip, of the type which are commonly used to aid recovery of patients with an injury, to prevent injury or further injury or to provide support during exertion. A skilled person is readily able to select an appropriate tape, examples of which include Kinesiology Tape, Kinesio Tex Tape; KT Tape; and RockTape.

In the embodiment shown in FIG. 1, elastic conductor 140 is glued onto tape substrate 120. By combining therapeutic tape 120 with elastic conductor 140 the inventors have created a device 100 capable of sensing change in geometry such as with stretch and elongation.

Elastic conductor 140 may be comprised of any suitable elastic material such as, a woven material; a non-woven material; a natural material; or a synthetic material or a blend thereof.

In one embodiment elastic conductor 140 comprises a conductive textile or any conductive material that changes resistance and/or capacitance as the tape stretches and bends. A suitable material for elastic elongate or sheet conductor comprises the product known as “stretch conductive fabric”. The stretch conductive fabric comprises a silver plated, polyamide containing elastic fibre fabric. While the antibacterial properties of silver make it attractive for use, other conductive metals may be substituted for silver. Additionally, other elastic polymeric materials may be used instead of a polyamide. Additionally, any conductive material which is capable of changing electrical properties during stretch or elongation may be used.

In another embodiment the electrical conductor may comprise or consist of graphene. The graphene can sprayed and/or painted on to tape substrate 120.

Graphene is a crystalline allotrope of carbon with two-dimensional properties. As graphene is stretched the molecules become less in contact with neighbouring molecules which causes the resistance to increase. The change in resistance may then be used to determine movement.

In another embodiment, the elastic two dimensional conductor comprises or consists of gold, silver, nickel, conductive carbon or any other suitable material capable of changing its electrical properties to detect deformation.

The elastic conductor 140 may be fixed to the tape substrate 120 using any suitable method such as, a gluing, screwing, nailing, stitching, riveting, chemical bonding, interweaving and coextrusion. Any suitable adhesive may be used as the glue. From the teaching herein a skilled person is readily able to join substrate 120 and conductor 140.

Elastic two dimensional conductor 140 may have any suitable shape. The skilled person will readily appreciate that the present invention is directed to monitoring movement and/or posture over a continuous area and therefore, conductor 140 should not be limited to recording data from a single point.

The shape of the elastic conductor 140 may be determined by the body part which is to be monitored. In one embodiment, elastic conductor 140 comprises an elongate shape. In another embodiment, elastic conductor comprises a broader shape, described herein as a sheet.

The elastic two dimensional conductor 140 allows an electrical current to flow across it, through it, or around it. The elastic elongate or sheet conductor 140 may also store a charge and may function as a capacitor. The current may be provided by a power supply 176 comprised on data collection unit (DCU) 160. The elastic conductor 140 produces deformation data comprising change in resistance and/or capacitance as the elastic elongate or sheet conductor changes geometry in response to movement.

Power supply 176 may also power the DCU 160. As shown in FIG. 1, the current is provided across conductor connectors 142, 144 which are connected to data collection unit connectors 162, 164. In another embodiment device 100 and DCU 160 comprises two separate power supplies. A sensor power supply (not shown) and a data collection power supply (not shown).

DCU 160 receives the deformation data and may store it on memory 170 as well as transmitting it wirelessly to personal computing device 200 via transceiver 172. This allows a user of the device 100 to effectively monitor the movement of the tape substrate 120 and thereby the body part to which it is affixed. The monitoring may be in real time.

The wireless connection 130 between DCU 160 and personal computing device 200 may be of any suitable type such as, a Bluetooth connection; a low energy Bluetooth connection; radio frequency connection; or wireless USB. The connection may be through transceiver 172 on DCU 160 and a linked transceiver on personal computing device 200.

When DCU 160 is connected to personal computing device 200 it may provide a visual representation of movement and position of the body on which tape substrate 120 is applied on the personal computing device 200. The visual representation may be in the form of an avatar, gauge, graph, chart, or any other visually meaning representation.

The DCU 160 may comprise or utilise GPS (Global Positioning System) data received from the personal computing device 200 to pattern movement behaviours to gain greater understanding of body movement across speeds and location of the user. For example, when a user is at work and predominately in negative body movement, this data can be used to help the user understand where the injury and/or pain is occurring.

The DCU 160 may detect and analyse movement patterns. For example DCU 160 may determine sitting, walking and/or running in conjunction with good or poor posture and/or body movement of the attached body part.

The DCU 160 may measure and learn movement patterns. The learning of movement patterns and their detection from received deformation data may be used to stop, warn and/or prevent harm to the user before they are aware of the potential damage.

The various functions of the DCU 160 are controlled by microcontroller 174. In another embodiment the DCU 160 may be controlled by a computing device similar to personal computing device 200.

In the embodiment shown in FIG. 1, DCU 160 is wearable and may be clipped onto an item of clothing, attached directly to the sensor tape, or affixed with adhesive to a body part. In another embodiment DCU comprises a strap and may be strapped to a body part.

In a preferable embodiment, DCU 160 is attached directly to substrate 120. The attachment may be by any suitable fixative such as, gluing, screwing, stitching, riveting, clipping, magnetic clipping, and chemical bonding. In another embodiment DCU 160 is attached to substrate 120 via insertion into a pouch (not shown).

Advantageously, the data collection unit 160 is small enough to be worn long term.

DCU 160 may further comprise one or more additional sensors 166. In the embodiment shown in FIG. 1 DCU 160 comprises temperature sensor 166a; three axis accelerometer 166b; a three axis gyroscope 166c; and a strain gauge 166d. A skilled person is readily able to select other suitable additional sensors for inclusion on DCU 160 or inclusion on substrate 120.

The sensor data collected from the one or more additional sensor 166 may also be stored on memory 170 or sent to personal computer device 200. In embodiments wherein analysis is being performed remotely or otherwise without input from a wearer, data from the one or more additional sensor 166 may be used to determine the type and extent of activities being performed.

In the embodiment shown in FIG. 1, DCU 160 comprises an alarm 168 for emitting an alert. In FIG. 1 the alarm 168 comprises a speaker that emits a sound when activated. In other embodiments, the alarm 168 may also comprise a light emitting diode or similar light source and/or a vibrating alarm. The vibrating alarm may emit a physical vibration that is readily felt by a user.

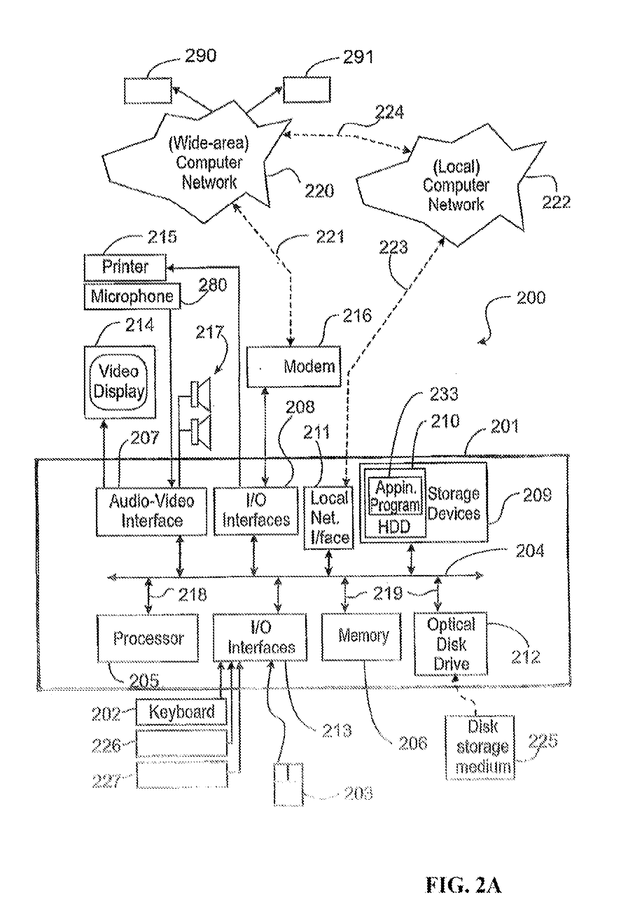

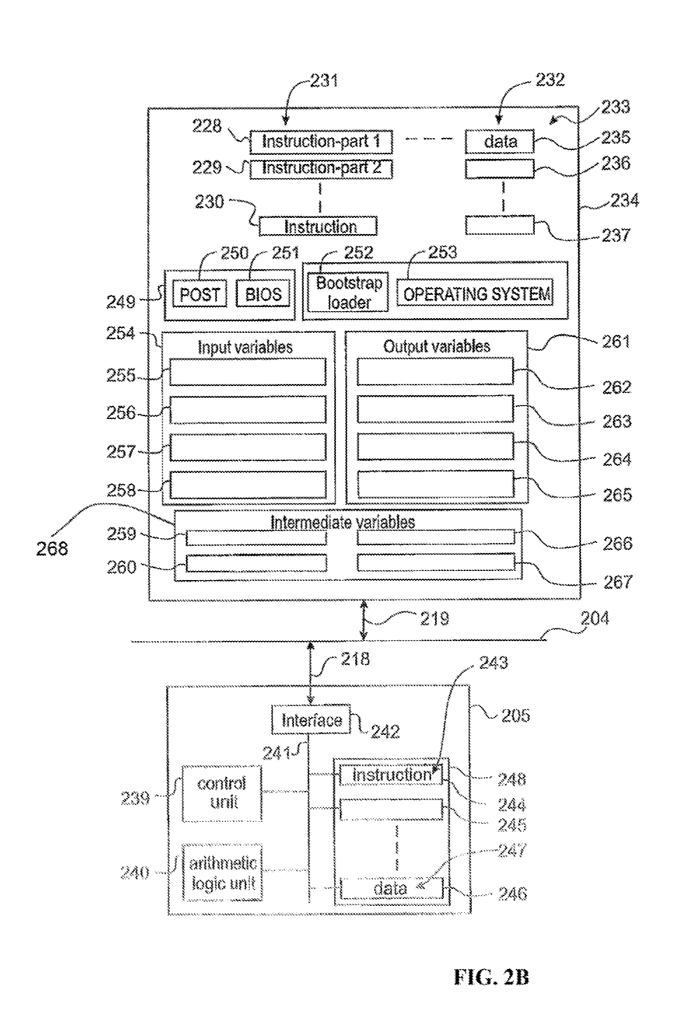

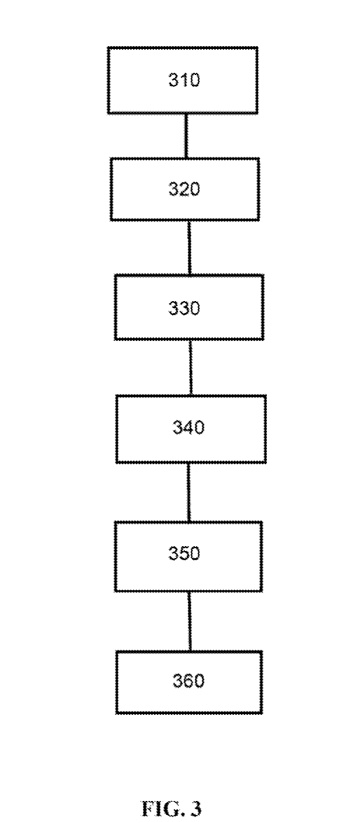

The alarm 168 may be activated when an undesired movement is detected. The detection may be when received deformation data matches reference deformation data associated with the undesired movement. The reference deformation data may be stored on memory 170 or a similar memory comprised in personal computing device 200 or a server computer 291 shown in FIG. 2A. As will be elucidated below, the data collection unit 160 may be connected wirelessly to the personal computing device 200 which may be connected to the server computer 291 through a network 220.

The alarm 168 may comprise a vibration actuated by a vibration motor (not shown) comprised in data collection unit 160. The vibration alarm is particularly advantageous because it provides physical feedback for users, warning them of times spent in poor posture or potential risks or reminders.

The deformation data may be analysed and/or viewed in detail on personal computing device 200 and used to provide patient feedback and monitoring to improve body position or posture. The personal computing device 200 may perform various analytical functions to provide useful graphical representations and/or summaries of data. The personal computing device 200 may then in turn send the data through a network 220 to a server computer 291 where a treating clinician can view the results and provide additional patient feedback. Data from multiple patients may also be used for research and analysis.

The deformation data may allow determination of change in geometry of the elastic two dimensional conductor 140. The change in geometry may comprise one or more of elongation and retraction.

The embodiment in FIG. 1 shows the elastic two dimensional conductor 140 to comprise a U-shape which comprises two proximally located ends 146, 148. Such shapes are advantageous in that the distance required for the connections between the DCU 160 and connections 142, 144 may be minimised. From the teachings herein, a skilled person is readily able to select other suitable shapes.

The shape may comprise a weave of conductive material in any shape or pattern such to collect required deformation data.

The shape may comprise one or more materials capable of changing electrical properties when deformed in a shape or pattern so as to collect required deformation data.

Examples of shapes comprising two or more proximally located ends include a U-shape; a V-shape; an arch; and an ogive.

The shape of conductor 140 may comprise two or more shapes comprising proximally located ends. Each of the two or more shapes may be connected in parallel or series. Each of the two or more shapes may be located side by side; arranged in a pattern or nested.

The shape may comprise an incomplete circle or oval. The incomplete circle or oval may comprise a missing portion of its circumference.

Examples of arch shapes include a triangular arch; a round arch; a rowlock arch; a segmental arch; an unequal round arch; a lancet arch; an equilateral pointed arch; a shouldered flat arch; a trefoil arch; a horseshoe arch; a three centred arch; an elliptical arch; an inflexed arch; an ogee arch; a reverse ogee arch; a tudor arch; a parabolic arch; a stilted arch; a bell arch; a cusped arch; and a Florentine arch. Examples of ogives are a standard ogive; an equilateral ogive; a lancet ogive; a dropped ogive and a Moorish ogive.

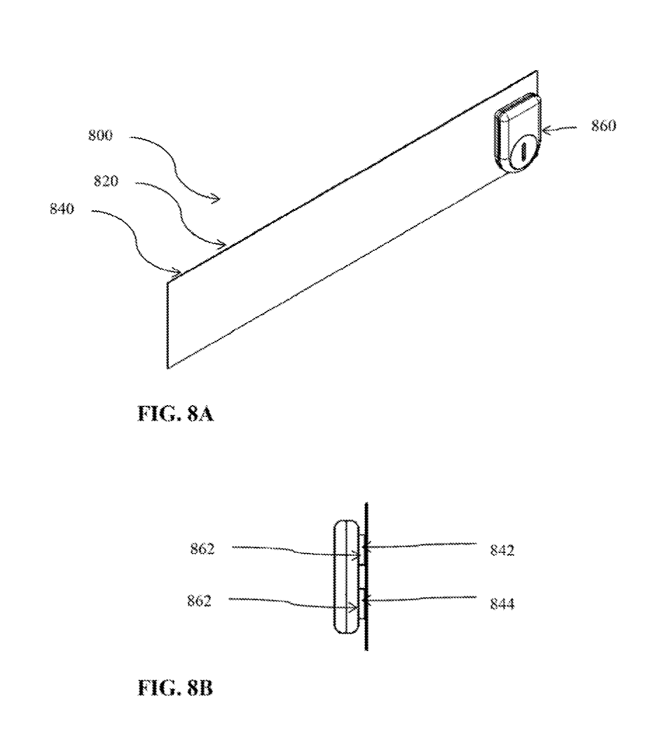

FIGS. 8 to 10 show another embodiment of a device 800 according to the invention. FIG. 8A is a perspective view showing a linear tape substrate 820 with data collection unit 860 located at one end. The side view of FIG. 8B partially shows conductor connectors 842, 844 and data collection unit connectors 862, 864 joined together to secure DCU 860 to substrate 820.

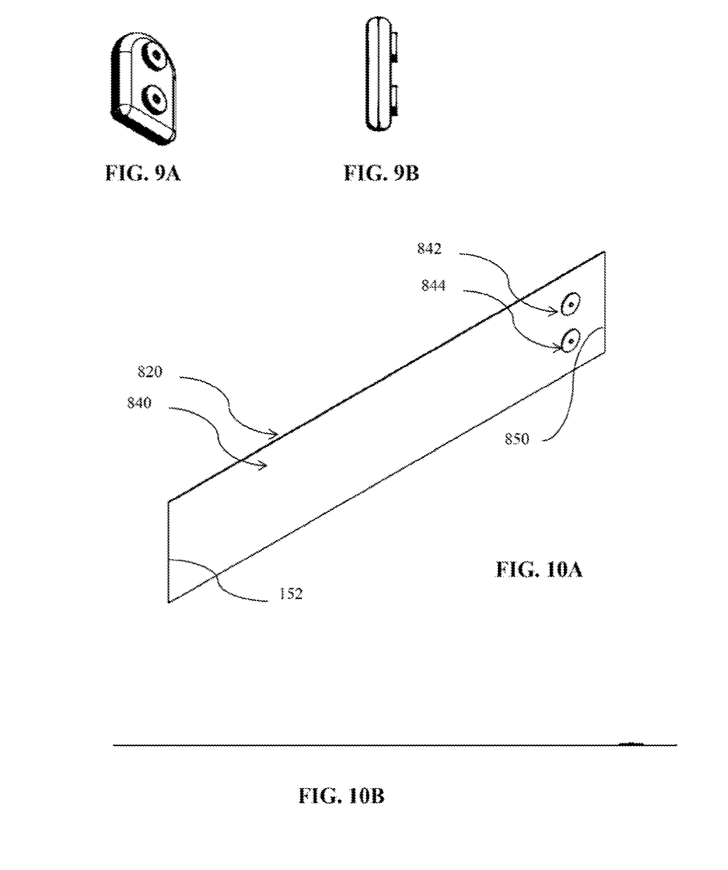

In the embodiment shown in FIGS. 8A and 8B, conductor connectors 842, 844 and data collection unit connectors 862, 864 comprise inter-fitting magnetic studs. The magnetic studs hold the DCU 860 in place and conduct electricity, providing the DCU 860 with a conduit to connect to the substrate 820. Better views of these inter-fitting magnetic studs are provided FIGS. 9A, 9B, 10A and 10B.

DCU 860 comprises a micro controller 874 in the form of an OLP425 available from ublox.

FIGS. 9A and 9B show perspective views of the DCU 860.

The top view of DCU 160 in FIG. 8A shows that part of the case can be removed to allow a battery, comprising part of power supply 876, to be removed.

In the embodiment shown in FIGS. 8 to 10, conductor 840 is disposed along the length of tape substrate 820 which comprises a sheet or strip shape. This elongate embodiment of tape substrate 820 and conductor 840 comprises opposed ends 850, 852. Both conductor connectors 842, 844 are disposed at connector opposed end 850.

One embodiment of a personal computing device 200 suitable for use in the present invention is shown in FIGS. 2A and 2B. In the embodiment of personal computing device 200 shown comprises a computer module 201 comprising input devices such as a keyboard 202, a mouse pointer device 203, a scanner 226, an external hard drive 227, and a microphone 280; and output devices including a printer 215, a display device 214 and loudspeakers 217. In some embodiments video display 214 may comprise a touch screen.

A Modulator-Demodulator (Modem) transceiver device 216 may be used by the computer module 201 for communicating to and from a communications network 220 via a connection 221. The network 220 may be a wide-area network (WAN), such as the Internet, a cellular telecommunications network, or a private WAN. Through the network 220, computer module 201 may be connected to other similar personal computing devices 290 or server computers 291. Where the connection 221 is a telephone line, the modem 216 may be a traditional “dial-up” modem. Alternatively, where the connection 221 is a high capacity (e.g.: cable) connection, the modem 216 may be a broadband modern. A wireless modem may also be used for wireless connection to network 220. A wireless transceiver (not shown) may also be used for wireless connection to DCU 160.

The computer module 201 typically includes at least one processor 205, and a memory 206 for example formed from semiconductor random access memory (RAM) and semiconductor read only memory (ROM). The module 201 also includes a number of input/output (I/O) interfaces including: an audio-video interface 207 that couples to the video display 214, loudspeakers 217 and microphone 280; an I/O interface 213 for the keyboard 202, mouse 203, scanner 226 and external hard drive 227; and an interface 208 for the external modem 216 and printer 215. In some implementations, modern 216 may be incorporated within the computer module 201, for example within the interface 208. The computer module 201 also has a local network interface 211 which, via a connection 223, permits coupling of the personal computing device 200 to a local computer network 222, known as a Local Area Network (LAN). The LAN may comprise connection with DCU 160 such as by wireless connection 130.

As also illustrated, the local network 222 may also couple to the wide network 220 via a connection 224, which would typically include a so-called “firewall” device or device of similar functionality. The interface 211 may be formed by an Ethernet circuit card, a Bluetooth wireless arrangement, including a Bluetooth low energy wireless arrangement or an IEEE 802.11 wireless arrangement or other suitable interface.

The I/O interfaces 208 and 213 may afford either or both of serial and parallel connectivity, the former typically being implemented according to the Universal Serial Bus (USB) standards and having corresponding USB connectors (not illustrated).

Storage devices 209 are provided and typically include a hard disk drive (HDD) 210. Other storage devices such as, an external HD 227, a disk drive (not shown) and a magnetic tape drive (not shown) may also be used. An optical disk drive 212 is typically provided to act as a non-volatile source of data. Portable memory devices, such optical disks (e.g.: CD-ROM, DVD, Blu-Ray Disc), USB-RAM, external hard drives and floppy disks for example may be used as appropriate sources of data to the personal computing device 200. Another source of data to personal computing device 200 is provided by the at least one server computer 291 through network 220.

The components 205 to 213 of the computer module 201 typically communicate via an interconnected bus 204 in a manner that results in a conventional mode of operation of personal computing device 200. In the embodiment shown in FIGS. 2A and 2B, processor 205 is coupled to system bus 204 through connections 218. Similarly, memory 206 and optical disk drive 212 are coupled to the system bus 204 by connections 219. Examples of personal computing devices 200 on which the described arrangements can be practiced include IBM-PC's and compatibles, Sun Sparc stations, Apple computers; smart phones; tablet computers or like a device comprising a computer module like computer module 201. It is to be understood that when personal computing device 200 comprises a smart phone or a tablet computer, display device 214 may comprise a touch screen and other input and output devices may not be included such as, mouse pointer device 201; keyboard 202; scanner 226; and printer 215.

FIG. 2B is a detailed schematic block diagram of processor 205 and a memory 234. The memory 234 represents a logical aggregation of all the memory modules, including the storage device 209 and semiconductor memory 206, which can be accessed by the computer module 201 in FIG. 2A.

The computational methods of the invention may be implemented using DCU 160 and/or personal computing device 200 wherein the computational methods may be implemented as one or more software application programs 233 executable within computer module 201. In particular, the steps of the methods of the invention may be effected by instructions 231 in the software carried out within the computer module 201

The software instructions 231 may be formed as one or more code modules, each for performing one or more particular tasks. The software 233 may also be divided into two separate parts, in which a first part and the corresponding code modules performs the method of the invention and a second part and the corresponding code modules manage a graphical user interface between the first part and the user.

The software 233 may be stored in a computer readable medium, including in a storage device of a type described herein. The software is loaded into the personal computing device 200 from the computer readable medium or through network 221 or 223, and then executed by personal computing device 200. In one example the software 233 is stored on storage medium 225 that is read by optical disk drive 212. Software 233 is typically stored in the HDD 210 or the memory 206.

A computer readable medium having such software 233 or computer program recorded on it is a computer program product. The use of the computer program product in the personal computing device 200 preferably effects a device or apparatus for implementing the methods of the invention.

In some instances, the software application programs 233 may be supplied to the user encoded on one or more disk storage medium 225 such as a CD-ROM, DVD or Blu-Ray disc, and read via the corresponding drive 212, or alternatively may be read by the user from the networks 220 or 222. Still further, the software can also be loaded into the personal computing device 200 from other computer readable media. Computer readable storage media refers to any non-transitory tangible storage medium that provides recorded instructions and/or data to the computer module 201 or personal computing device 200 for execution and/or processing. Examples of such storage media include floppy disks, magnetic tape, CD-ROM, DVD, Blu-ray Disc, a hard disk drive, a ROM or integrated circuit, USB memory, a magneto-optical disk, or a computer readable card such as a PCMCIA card and the like, whether or not such devices are internal or external of the computer module 201. Examples of transitory or non-tangible computer readable transmission media that may also participate in the provision of software application programs 233, instructions 231 and/or data to the computer module 201 include radio or infra-red transmission channels as well as a network connection 221, 223, 224, to another computer or networked device 290, 291 and the Internet or an Intranet including email transmissions and information recorded on Websites and the like.

The second part of the application programs 233 and the corresponding code modules mentioned above may be executed to implement one or more graphical user interfaces (GUIs) to be rendered or otherwise represented upon display 214. Through manipulation of, typically, keyboard 202, mouse 203 and/or screen 214 when comprising a touch screen, a user of personal computing device 200 and the methods of the invention may manipulate the interface in a functionally adaptable manner to provide controlling commands and/or input to the applications associated with the GUI(s). Other forms of functionally adaptable user interfaces may also be implemented, such as an audio interface utilizing speech prompts output via loudspeakers 217 and user voice commands input via microphone 280. The manipulations including mouse clicks, screen touches, speech prompts and/or user voice commands may be transmitted via network 220 or 222.

When the computer module 201 is initially powered up, a power-on self-test (POST) program 250 may execute. The POST program 250 is typically stored in a ROM 249 of the semiconductor memory 206. A hardware device such as the ROM 249 is sometimes referred to as firmware. The POST program 250 examines hardware within the computer module 201 to ensure proper functioning, and typically checks processor 205, memory 234 (209, 206), and a basic input-output systems software (BIOS) module 251, also typically stored in ROM 249, for correct operation. Once the POST program 250 has run successfully, BIOS 251 activates hard disk drive 210. Activation of hard disk drive 210 causes a bootstrap loader program 252 that is resident on hard disk drive 210 to execute via processor 205. This loads an operating system 253 into RAM memory 206 upon which operating system 253 commences operation. Operating system 253 is a system level application, executable by processor 205, to fulfill various high level functions, including processor management, memory management, device management, storage management, software application interface, and generic user interface.

Operating system 253 manages memory 234 (209, 206) in order to ensure that each process or application running on computer module 201 has sufficient memory in which to execute without colliding with memory allocated to another process. Furthermore, the different types of memory available in the personal computing device 200 must be used properly so that each process can run effectively. Accordingly, the aggregated memory 234 is not intended to illustrate how particular segments of memory are allocated, but rather to provide a general view of the memory accessible by computer module 201 and how such is used.

Processor 205 includes a number of functional modules including a control unit 239, an arithmetic logic unit (ALU) 240, and a local or internal memory 248, sometimes called a cache memory. The cache memory 248 typically includes a number of storage registers 244, 245, 246 in a register section storing data 247. One or more internal busses 241 functionally interconnect these functional modules. The processor 205 typically also has one or more interfaces 242 for communicating with external devices via the system bus 204, using a connection 218. The memory 234 is connected to the bus 204 by connection 219.

Application program 233 includes a sequence of instructions 231 that may include conditional branch and loop instructions. Program 233 may also include data 232 which is used in execution of the program 233. The instructions 231 and the data 232 are stored in memory locations 228, 229, 230 and 235, 236, 237, respectively. Depending upon the relative size of the instructions 231 and the memory locations 228-230, a particular instruction may be stored in a single memory location as depicted by the instruction shown in the memory location 230. Alternately, an instruction may be segmented into a number of parts each of which is stored in a separate memory location, as depicted by the instruction segments shown in the memory locations 228 and 229.

In general, processor 205 is given a set of instructions 243 which are executed therein. The processor 205 then waits for a subsequent input, to which processor 205 reacts by executing another set of instructions. Each input may be provided from one or more of a number of sources, including data generated by one or more of the input devices 202, 203, or 214 when comprising a touch screen, data received from an external source across one of the networks 220, 222, data retrieved from one of the storage devices 206, 209 or data retrieved from a storage medium 225 inserted into the corresponding reader 212. The execution of a set of the instructions may in some cases result in output of data. Execution may also involve storing data or variables to the memory 234.

The disclosed arrangements use input variables 254 that are stored in the memory 234 in corresponding memory locations 255, 256, 257, 258. The described arrangements produce output variables 261 that are stored in the memory 234 in corresponding memory locations 262, 263, 264, 265. Intermediate variables 268 may be stored in memory locations 259, 260, 266 and 267.

The register section 244, 245, 246, the arithmetic logic unit (ALU) 240, and the control unit 239 of the processor 205 work together to perform sequences of micro-operations needed to perform “fetch, decode, and execute” cycles for every instruction in the instruction set making up the program 233. Each fetch, decode, and execute cycle comprises:
(a) a fetch operation, which fetches or reads an instruction 231 from memory location 228, 229, 230;
(b) a decode operation in which control unit 239 determines which instruction has been fetched; and
(c) an execute operation in which the control unit 239 and/or the ALU 240 execute the instruction.

Thereafter, a further fetch, decode, and execute cycle for the next instruction may be executed. Similarly, a store cycle may be performed by which the control unit 239 stores or writes a value to a memory location 232.

Each step or sub-process in the methods of the invention may be associated with one or more segments of the program 233, and may be performed by register section 244-246, the ALU 240, and the control unit 239 in the processor 205 working together to perform the fetch, decode, and execute cycles for every instruction in the instruction set for the noted segments of program 233.

One or more other computers 290 may be connected to the communications network 220 as seen in FIG. 2A. Each such computer 290 may have a similar configuration to the computer module 201 and corresponding peripherals.

One or more other server computer 291 may be connected to the communications network 220. These server computers 291 respond to requests from the personal computing device or other server computers to provide information.

Data collected by the data collection unit 160 may be transmitted and/or stored on one or more server computer 291. The stored data may be viewed by a health or professional, performance professional or by the wearer. The health professional, performance professional or wearer may have access to an interface on a personal computing device 200. The health professional or performance professional can interpret the data which may be shown in a table, graph, chart or other visual method and provide feedback to the wearer of the device.

The computational methods of the invention may alternatively be implemented in dedicated hardware such as one or more integrated circuits performing the functions or sub functions of the described methods. Such dedicated hardware may include graphic processors, digital signal processors, or one or more microprocessors or microcontroller, such as microprocessor 174, 874, and associated memories such as memory 170.

The invention also provides a method 300 for recording movement and/or posture in a continuous area. The method comprises applying 310 an elastic therapeutic tape substrate 120 to a body part. As detailed above, the elastic therapeutic tape substrate 120 comprises an elastic two dimensional conductor 140 along which an electrical current can flow and which covers an area of the elastic therapeutic tape substrate 120.

Method 300 also includes producing 320 deformation data comprising change in resistance and/or capacitance as the elastic two dimensional conductor changes geometry in response to movement to thereby record the movement in the continuous area.

Method 300 may further comprise recording 330 deformation data to the data collection unit 160.

Method 300 may further comprise joining 340 two or more connectors 142, 144 on the elastic two dimensional conductor to respective two or more connectors 162, 164 on the data collection unit 160.

Method 300 may further comprise receiving 350 data from one or more additional sensor 166.

Method 300 may further comprise activating 360 an alert when an undesired movement or series of movements is detected from the received deformation data.

A plurality of tape substrates 120, each comprising a two dimensional conductor 140, may be applied at different positions on one or more body parts. Each of the plurality of substrates 120 may comprise respective DCUs 160 or one common DCU may be connected to each of the plurality of substrates 120.

The invention also provides a kit 400 (not shown) comprising one or more devices 100, 800 according to the first aspect including one or more data collection units 160, 860. The kit of the invention may also comprise a plurality of tape substrates 120, 820 each comprising a two dimensional conductor 140, 840 for application at different positions on one or more body parts. As noted above, each of the plurality of substrates 120, 820 may comprise respective DCUs 160, 860 or one common DCU 160, 860 may be connected to each of the plurality of substrates 120, 820.

The device 100, 800 method 300 and kit 400 may be for use or when used to measure activity and/or assess biomechanics for prevention, reduction and/or rehabilitation of an injury, disease or other condition or improvement or modification of athletic performance or posture.

The disease may be a disease such as a degenerative disease or compromised mobility. The degenerative disease may comprise one or more of Alzheimer's disease; Amyotrophic Lateral Sclerosis (ALS), a.k.a., Lou Gehrig's Disease; Osteoarthritis; Atherosclerosis; Chronic obstructive Pulmonary Disease (COPD); Chronic traumatic encephalopathy; Diabetes; Ehlers-Danlos Syndrome; Essential tremor; Friedreich's ataxia; Huntington's Disease; Marfan's Syndrome; Multiple sclerosis; Multiple system atrophy; Muscular dystrophy; Niemann Pick disease; Osteoporosis; Parkinson's Disease; Progressive supranuclear palsy; Rheumatoid Arthritis; Motor neuron Conditions; Metabolic Muscle Conditions; Conditions of the peripheral nerve; Conditions of the Neuromuscular Junction; Neuromuscular Myopathies; Chronic Pain; Chronic Swelling; Bursitis; Fibromyalgia; Back Pain; Scoliosis; Tendonitis; Degenerative Disk Disease.

The device 100, 500, 800, method 300 and kit 400 may also be used to reduce occupational health and safety issues by monitoring movements of high risk or injured employees (e.g. Lifting technique, sustained postures, postures of increased risk) and/or to improve research techniques when reviewing movement in everyday life and activity.

The device 100, 800, method 300 and kit 400 may also be used to provide real time information for a treating clinician to review a patients' progress and rehabilitation to greater tailor treatment or intervention and/or provide postural information over the course of hours or days for the user, Occupational Health & Safety representative of companies and treating therapists.

The device 100, 800, method 300 and kit 400 may also be used to provide feedback for minimising posture or pressure related problems in the elderly and persons of compromised mobility (e.g. Children with Cerebral Palsy, the elderly, patients with spinal compromise, other movement based disorders).

The device 100, 800, method 300 and kit 400 of the invention may also provide biofeedback for neurological compromised patients (e.g. Stroke or Parkinson's disease) or any person where movement quality may be affected and or falls prevention through early and high quality feedback related to posture.

The device 100, 800, method 300 and kit 400 may also aid in the prevention of injury as well as rehabilitation of an existing injury and/or aid in the identification of potential injury causing events.

In yet another embodiment of any above aspect, the body part may comprise a human or non-human body part. The non-human body part may comprise a performance animal body part such as, an equine or a canine body part.

The following non-limiting examples illustrate the device 100, 800 and methods 300 of the invention. These examples should not be construed as limiting: the examples are included for the purposes of illustration only. The device 100, 800 discussed in the Examples will be understood to represent an exemplification of the invention.

### EXAMPLES

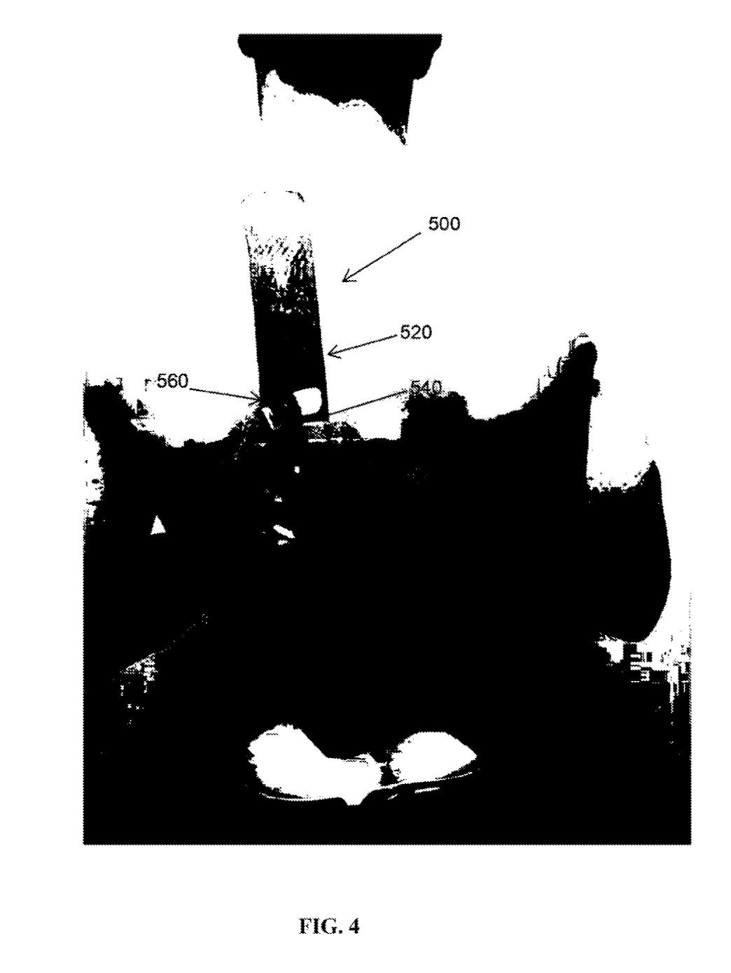

FIGS. 4 and 5 show prototype devices 500 according to the invention. The prototype device or E-Tape 500 comprises Kinesio Tex Tape as the elastic therapeutic tape 520 in combination with an elastic conductor 540 comprising a material called “Stretch Conductive Fabric” purchased from http://www.lessemf.com/fabric.html which contains silver plated 76% nylon and 24% elastic. The fabric 540 (obscured under tape 520) was cut into a thin lengthways piece and then cut into a V or U shape which enables a complete circuit in which current can flow along the length of the sensor. The conductive material 540 was glued, with flexible craft glue, onto the elastic therapeutic tape 520, enabling the conductive fabric 540 to stretch when the tape 520 stretches.

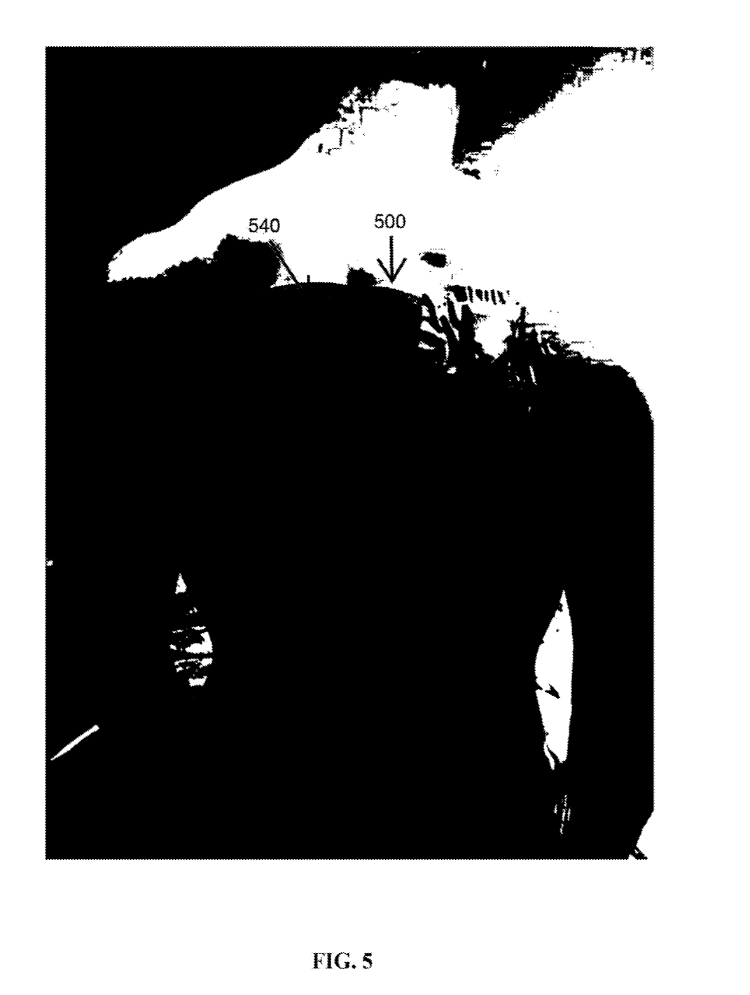

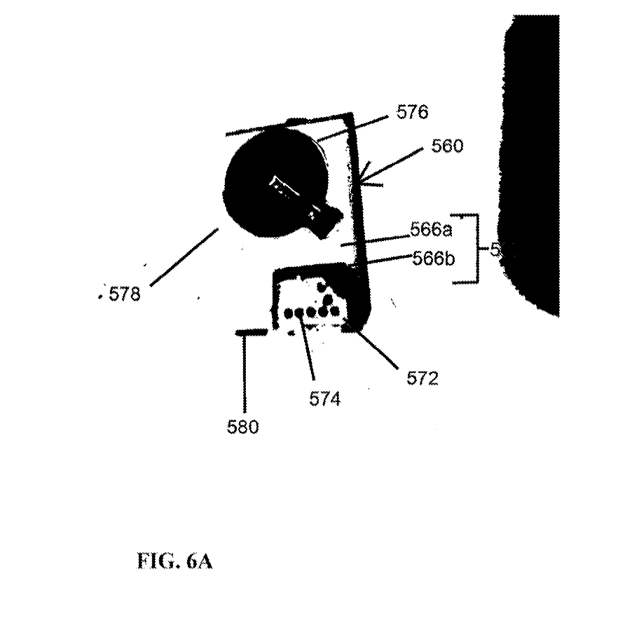

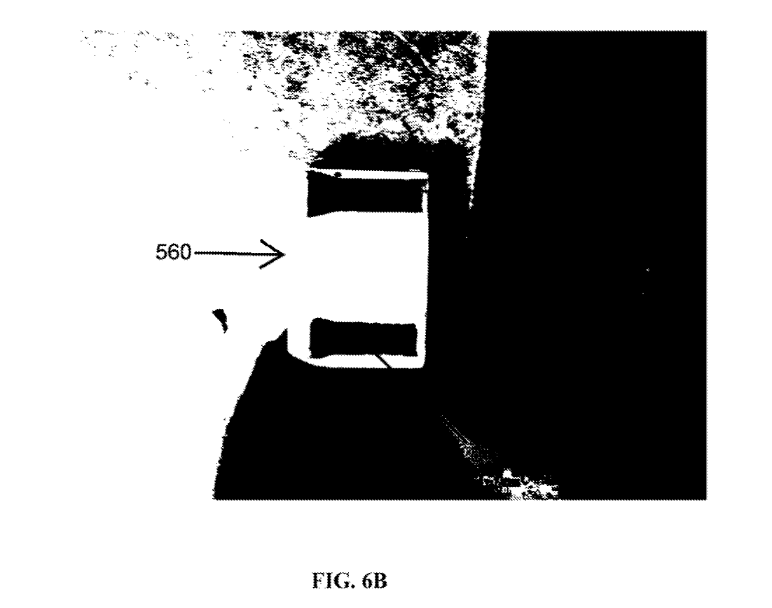

The prototype DCU 560 is affixed with tape in the embodiment shown in FIGS. 4 and 5. As the tape obscures the prototype DCU in FIGS. 4 and 5, FIGS. 6A and 6B show front and rear views of the prototype DCU 560. The Prototype DCU 560 comprises a battery 576; a microcontroller 574 with on-board sensors 566; case 578; memory 570; a transceiver 572; and conductive pads/clips 562, 564 for connection to E-Tape 400. In the prototype shown in FIGS. 6A and 6B, on-board sensors 566 comprise a temperature sensor 566a (obscured by case 178) and 3 axis accelerometer 566b (obscured by case 578).

Prototype DCU 560 shown in FIGS. 6A and 6B also comprises connector 580 which may be used to update or add software to DCU 560. Connector 580 may also be used for connecting to connections 542, 544 on tape 540.

The prototype 560 utilises a purchased Bluetooth low energy microcontroller 574 (OLP425) from Connect Blue. The microcontroller 574 measures the change in geometry of the E-Tape 500 by placing a voltage (or a timed pulse of voltage) through the tape 500 and then measuring the change in voltage across the E-Tape 500. The microcontroller 574 then uses an analogue to digital convertor to create a digital signal to be sent to an personal computing device 200, which stores the deformation data and provides feedback to the patient.

The DCU 560 connects to the tape 500 through two conductors 562, 564, which are either: Conductive plates on the exterior casing which are in line with the conductive material of the E-tape; or conductive clips; or conductive adhesive.

The DCU 560 prototypes thus far have been encased in epoxy or plastic with the intent of being robust and waterproof.

Various software modifications were made to the microcontroller to enable the required functionality.

### Mobile Device

Software has been developed for an iPhone to function as the personal computing device 200, which connects to the DCU 160, 560 and gives visual feedback to the user. Data from the DCU 160, 560 is analysed by the mobile device 200 to provide the desired feedback to the user. Feedback thus far has been achieved visually, however this will be expanded on this in the future to provide auditory, vibratory, visual, graphical, and historical summaries

### Central Server

A cloud based server 291 stores data sent to it via the user's personal computing device 200. Currently, ‘Parse’, a mobile database provider, is being used to capture and store collected data.

### Software Flow Chart

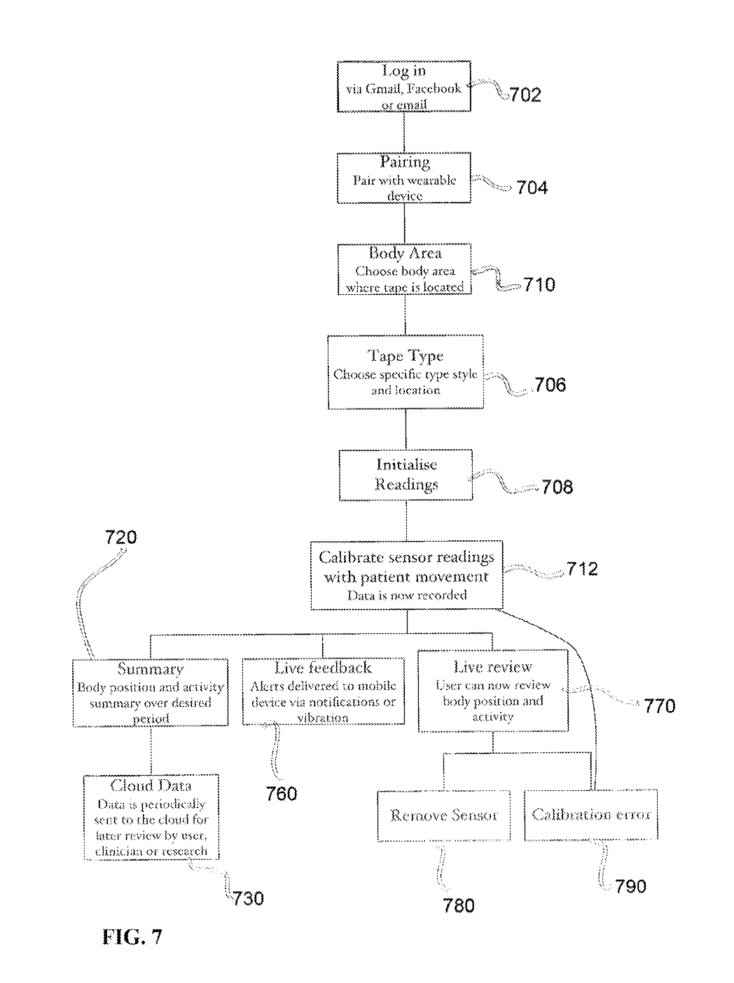

FIG. 7 shows one embodiment of a method 700 according to the invention. In step 702 a user logs in to the software program installed on personal computing device 200 which is used to interface with DCU 160, 560, 860. In step 704, Personal Computing Device 200 pairs with DCU 160, 560, 860. In step 710 a body area is chosen in which to apply the device 100, 500, 800. In step 706 the tape type is selected (appropriate length, width and elasticity of device 100, 500, 800) and applied to the chosen body area. Step 710 also requires the DCU 160, 560, 860 to be connected to the tape device 100, 500, 800 and affixed to the body. In step 708 the readings from device 100, 500, 800 are initialised and in step 712 the sensor readings are calibrated with patient movement. From this point on data may now be recorded by either DCU 160, 560, 860, personal computing device 200 or server computer 291.

In step 720 deformation data is produced, which may include a summary of body position and activity over a desired period. This data may be stored on the DCU 160, 560, 860 or on a Personal Computing Device 200.

In step 730 the data is sent from Personal Computing Device 200 to server 291 or to other cloud locations for later review by a user, clinician, heath care professional, performance professional, or researcher. This data can be viewed on a personal computing device 200, which may be remote and/or disconnected from any DCU 160, 560, 860.

Step 760 an alert may be activated. The activation of the alert may be in response to live feedback delivered to personal computing device 200. The alert may be delivered via a notification such as, by vibration and/or auditory alert and/or visual alert. The alert, and activation of alert, may emanate from Personal Computing Device 200 or DCU 160, 560, 860.

In step 770 a user may conduct a live review, which allows review of body position and activity.

In step 780 the sensor may be removed and in step 790 the user may be alerted or become aware of a calibration error, which may result in the need to repeat one or all of steps 702, 704, 710, 706, 708, 712, 770, 790.

### Future Designs

A conductive material/yarn 140 of some type will be woven, laminated, coextruded or otherwise combined into a tape 120, 520, 820 to create a complete device 100, 500, 800 that looks and feels like conventional elastic therapeutic tape. Multiple lengths and widths of tape will be manufactured for multiple purposes. Different weave/lamination types, shapes, patterns, density and thickness will be examined for maximum efficiency and effectiveness for each desired purpose.

Multiple sensor arrays will be incorporated within a single device 100, 500, 800 to increase relevant data, increase data resolution and more effectively target specific areas of the body.

New conductive materials will be included into future prototypes, which include resistive materials, thread/yarn, polymers and conductive adhesives.

Also conductive thread will be woven into the elastic tape to create a pattern that when stretched changes the electrical resistance; conductive Rubber/polymers will be created through the addition of a conductive compound making the whole material or parts of the material conductive.

The conductive material comprising conductor 140, 540, 840 may be woven into tape substrate 120, 520, 820. The weave may comprise any pattern including U-shaped and zig-zag patterns.

The shape may comprise a weave of conductive material in any shape or pattern such to collect required deformation data.

The shape may comprise one or more materials capable of changing electrical properties when deformed in a shape or pattern so as to collect required deformation data.

Additionally, conductive polymer fabric will be applied to various fabric/materials. The coating does not affect the fabric/materials inherent ability to stretch and bend. The coating adds electrical conductivity/properties to the fabric which varies as the fabric/material is deformed or stretched.

Capacitive materials may also be incorporated.

Additionally, dielectric Elastomers/Piezoelectric Polymer in which an elastomeric film is coated on both sides with a dielectric material, creating a capacitor, which varies capacitance or creates a voltage when the elastomer is displaced, may be used.

The conductive material will potentially extend to the top surface of the device 100, 500, 800 enabling the DCU 160, 560, 860 to be interfaced with the surface of the tape 120, 520, 820 however it may also extend through all portions of the tape 120, 520, 820 or be of laminated construction. Attaching the DCU 160, 560, 860 can potentially occur through any number of methods: a pouch integrated into the E-Tape Device 100, 500, 800; adhesive tabs (conductive and/or non-conductive); clip mechanism (conductive and/or non-conductive); plug/s (conductive and/or non-conductive); pin/s (conductive and/or non-conductive); Magnetic attachments; and any combination of the above.

The DCU 160, 560, 860 may also incorporate improvements to enhance the overall usability of the device such as: auditory alarm; vibratory motor; Lights or LED's for visual output; rechargeable; ability to connect to multiple pieces of E-Tape device 100, 500, 800 using various connection methods listed previously; connection to multiple DCUs 100, 500, 800 placed over the body.

Software will be further developed to provide a seamless user interface with access to graphical and numerical data including:

Accelerometer data may be provided to determine the type and extent of activities being conducted by the patient including data analysis, output, feedback and user interface

Temperature sensor data analysis, output, feedback and user interface may also be provided.

The invention may also provide: E-Tape device 100, 500, 800 data analysis, output, feedback and user interface; access to data analysis, output, feedback and user interface for clinicians; access to data for purposes of research; access and storage of data for individual patients; cross analysis of some (or all) of the items above; and summary information (i.e. graphical) for patients and clinicians.

1. A device for recording movement and/or posture in a continuous area comprising:
an elastic therapeutic tape substrate for application to a body part, the tape substrate comprising an elastic two dimensional conductor along which an electrical current can flow to produce deformation data comprising change in resistance and/or capacitance as the elastic two dimensional conductor changes geometry in response to movement.

2. The device according to claim 1 wherein the device further comprises a data collection unit which receives the deformation data.

3. The device according to claim 2 wherein the elastic two-dimensional conductor comprises one or more connectors for joining to respective one or more connectors comprised on the data connection unit.

4. The device according to claim 2 wherein the data collection unit further comprises one or more additional sensor.

5. The device according to claim 2 wherein the data collection unit further comprises an alert.

6. The device according to claim 5 wherein the alert is activated when an undesired movement is detected from the received deformation data.

7. The device according to claim 6 wherein the undesired movement may be detected when received deformation data matches reference deformation data associated with the undesired movement.

8. A method for recording movement and/or posture in a continuous area comprising;
applying an elastic therapeutic tape substrate to a body part, the elastic therapeutic tape substrate comprising an elastic two dimensional conductor along which an electrical current can flow; and
producing deformation data comprising change in resistance and/or capacitance as the elastic two dimensional conductor changes geometry in response to movement to thereby record the movement in the continuous area.

9. The method according to claim 8 further comprising joining one or more connectors on the elastic two-dimensional conductor to respective one or more connectors on a data collection unit.

10. The method according to claim 9 further comprising recording deformation data on the data collection unit.

11. The method according to claim 10 further comprising receiving data from one or more additional sensor.

12. The method according to claim 10 further comprising activating an alert when an undesired movement is detected from the received deformation data.

13. The method according to claim 12 wherein the detection of the undesired movement comprises matching received deformation data with reference deformation data associated with the undesired movement.

14. The method according to claim 13 wherein the reference deformation data is stored on a memory comprised in the data collection unit or a memory comprised on a personal computing device or a server computer.

15. A kit comprising one or more devices according to claim 1.

16. A system for recording movement and/or posture in a continuous area comprising;
an elastic therapeutic tape substrate for application to a body part; and
an elastic two dimensional conductor comprised on the elastic therapeutic tape substrate along which an electrical current can flow for producing deformation data comprising change in resistance and/or capacitance as the elastic two dimensional conductor changes geometry in response to movement.

17. The system according to claim 16 further comprising a data collection unit for receiving the deformation data.

18. The system according to claim 17 further comprising respective connectors on the elastic two-dimensional conductor and the data collection unit for interconnection.

19. The system according to claim 16 further comprising one or more additional sensor for generating additional data.

20. The system according to claim 17 further comprising an alert for notifying when an undesired movement is detected from the received deformation data.
